(19) United States
(12) Patent Application Publication
(10) Pub. No.: US 2023/0021831 A1
(43) Pub. Date: Jan. 26, 2023

(54) AUTOINJECTOR WITH DISCHARGE DETECTION

(71) Applicant: Ypsomed AG, Burgdorf (CH)

(72) Inventors: Gabriel Kalbermatter, Burgdorf (CH); Leos Urbanek, Bern (CH); Markus Tschirren, Burgdorf (CH); Jürg Hirschel, Bern (CH); Martin Brügger, Bolligen (CH)

(21) Appl. No.: 17/952,986

(22) Filed: Sep. 26, 2022

Related U.S. Application Data
(63) Continuation of application No. PCT/EP2021/057168, filed on Mar. 22, 2021.

(30) Foreign Application Priority Data
Mar. 26, 2020 (CH) 00362/20

(51) Int. Cl. A61M 5/20; A61M 5/32; A61M 5/24

(52) U.S. Cl. CPC A61M 5/2033; A61M 5/326; A61M 5/3202; A61M 5/24; A61M 2005/2013; A61M 2005/206; A61M 2005/2492

(57) ABSTRACT

An autoinjector includes a housing, a product container, a torsion spring, a drive element, and a propulsion element. In order to discharge liquid out of the product container, the torsion spring rotates the drive element, and the rotating drive element produces a propulsive movement of the propulsion element and of a piston in the product container. A rotation sensor is configured for an alternating continuous detection of at least two rotational positions per revolution of the drive element during the discharge process, and a processor unit is configured for determining the axial position of the piston in the product container from the detected rotational positions.

### CROSS REFERENCE TO RELATED APPLICATIONS

This application claims priority to International Patent Application No. PCT/EP2021/1057168, filed Mar. 22, 2021, entitled “AUTOINJECTOR WITH DISCHARGE DETECTION,” and which in turn claims priority to Swiss Patent Application No. 00362/20, filed Mar. 26, 2020, entitled “AUTOINJECTOR WITH DISCHARGE DETECTION”, each of which is incorporated by reference herein, in the entirety and for all purposes.

### TECHNICAL FIELD

The present disclosure relates to the field of medical injection devices for administering liquid substances.

### BACKGROUND

Injection devices or injection apparatuses for the simplified administration of a substance include, inter alia, so-called autoinjectors, which may have an energy store to allow the discharge process to be carried out automatically, e.g., without a force to be supplied or exerted externally by a user. The energy store, advantageously, stores the energy required for an automatic substance delivery in mechanical form. Such an energy store can be a spring that is installed in a tensioned state in the injection device and delivers energy by expansion. The energy may be delivered to a piston rod or a pressure element, which pushes a piston into a product container. The energy store may also be provided in order to automate the process of injecting an injection needle. Alternatively, the injection process can take place manually, e.g., exclusively by a user, without using energy stored in the injection device for this purpose.

The injection device can include a product container holder for receiving a product container, where the product container can be held in the product container holder radially, axially, and may also in a rotationally-fixed manner. The product container holder can be connected to the housing of the injection device in an axially- and/or rotationally-fixed manner or can be moved relative to the housing during an injection and/or needle retraction process. The product container can be a carpule for the one-time or repeatedly detachable connection to disposable injection needles, or a disposable, ready-to-use syringe with a non-detachably connected injection needle. The product container may have a hollow cylindrical product container portion which displaceably mounts a piston or plunger. The piston can form a sealing gap with the inner circumference of the product container portion and can be displaced in a distal direction by means of a piston rod in order to dispense product from the product container via the injection needle.

The injection device can have a needle protective sleeve, which, after injection has taken place, projects distally beyond the distal end of the injection needle or is displaced relative to the housing into this position while expanding a needle protective sleeve spring, in order to prevent accidental access to the injection needle and thereby reduce the risk of injury. In an autoinjector, the needle protective sleeve can also serve as a trigger element for triggering the product discharge process, wherein the needle protective sleeve is displaced relative to the housing in the proximal direction for this purpose. Alternatively, the triggering of the autoinjector can be achieved by actuating a trigger button of the autoinjector, wherein the needle protective sleeve serves at least as a visual protection before the autoinjector is used.

Patent application WO 2016/205963 describes an exemplary autoinjector including a housing with a longitudinal axis, a trigger apparatus, a product container arranged in an axially-fixed manner in the housing. The autoinjector further includes a needle protective sleeve which is displaceable in a longitudinal direction between a proximal and a distal position and is coupled to a needle protection spring. A feedback apparatus with a stop element accelerated towards a stop by the needle protection spring serves to generate an acoustic signal after a specific amount of substance has been delivered. A spiral spring or mainspring, in which energy for the automatic discharge process of product can be stored, may be coupled to the trigger apparatus, where a first end of the spiral spring may be connected to the housing, and a second end of the spiral spring may be connected in a rotationally-fixed manner to a drive element, arranged coaxially with the longitudinal axis, in the form of a rotating, threaded rod. The threaded rod may engage via a thread in a propulsion member, which is not rotating in the housing, in the form of a sleeve-shaped piston rod which, when displaced in the distal direction, moves the plunger of the product container along with it at an at least approximately constant discharge rate.

Patent application WO 2017/097507 discloses an autoinjector with a pretensioned spiral spring, a drive element, and a piston rod, which is rotated by the drive element connected to the spiral spring and, in the process, axially displaces a piston in a syringe body. The rotation of a signaling element at the beginning of a discharge process is detected by closing an electrical contact with two contact points, either via a movement of a strip electrically insulating the contact points or via a relative movement of the contact points of a mechanical or magnetic switch, thereby activating a sensor unit of the autoinjector. Said sensor unit determines information for the discharge process, e.g., a number of revolutions of the signaling element, which can subsequently be compared to an already known number of revolutions.

Patent application WO 2019/129622 describes a manually-operated injection pen with dose selection, including a drive sleeve which is coupled to a piston rod in a rotationally-fixed and axially-displaceable manner and is rotated by a torsion spring. An optical rotation sensor detects the rotation of the drive sleeve by selectively radially reflecting, from an inner side of the drive sleeve, a light beam deflected in a prism. A discharged dose can be deduced from the number of registered reflections.

Patent application WO 2019/209491 describes a manual injection pen with variable dosage, including a capacitive, piezoelectric, or resistive expansion sensor for detecting an expansion of a bending beam in the form of an arm of a printed circuit board. This expansion may be induced by the rotational movement of a gear wheel. Each individual tooth of the gear wheel may press one end of the axially-oriented arm radially outwards so that the resulting mechanical expansion can be detected.

Patent application WO 2017/129314 describes an autoinjector with a detector for detecting a start of a discharge process based upon a movement of the needle protective sleeve in the proximal direction. By means of the detection, a communications unit of the autoinjector is activated.

Patent application WO 2018/069150 discloses an autoinjector with a controller for detecting the end of a discharge stroke based upon the inductance of a conductive helical or compression spring that expands during the discharge process and drives a piston sleeve. The inductance may be determined via the resonant frequency of an LC resonant circuit including the compression spring. The autoinjector includes an electronic indicator for signaling an injection end to the user, where this point in time can be an adjustable holding time after the detection of the discharge end.

### SUMMARY

This disclosure describes examples of an autoinjector with a torsion spring drive for automatically discharging a liquid from a product container, which may make it possible to determine an axial position of a piston in the container in a precise and/or energy-saving manner.

An autoinjector according to the disclosure may include a housing with a longitudinal axis, a product container, a torsion spring pretensioned for the one-time discharge of a maximum content of the product container, a drive element, and a propulsion element. For discharging a maximum or at least a predetermined amount of liquid from the product container, the torsion spring rotates the drive element about the longitudinal axis, and the rotating drive element may produce a propulsive movement of the propulsion element in order to displace a piston in the product container. The autoinjector may further include a rotation sensor for the alternating and continuous detection of at least two rotational positions per revolution of the drive element during the discharge process, as well as a processor unit for determining an axial piston position of the piston in the product container or a residual volume in the product container from the successively detected rotational positions. According to the disclosure, a rotation of a drive member caused directly by the torsion spring during the discharge process is measured continuously and with a resolution of half a revolution or less, and the propulsion and the piston position are determined therefrom. As a result of the improved resolution, the residual volume or the amount of medication discharged may be determined precisely, which may be important particularly in cases in which the discharge process does not proceed as planned, or is even interrupted by the user. The progress of a discharge process may be detected in real time, and in particular, fluctuations in the propulsion speed may be detected without delay; meaningful conclusions regarding a plunger siliconization in the context of quality monitoring in the case of aged autoinjectors may likewise be derived. This knowledge, as well as further injection data, may be subsequently fed to a central evaluation system for comparison to one another and to reference values in the context of a comprehensive monitoring of a fleet of autoinjectors.

In some embodiments, the propulsion element may have a non-rotationally symmetrical cross-section with an axial guide element in the form of a groove, plane, or rib parallel to the longitudinal axis, via which the propulsion element is guided, axially, linearly through the housing or a guide element, which may be accommodated in a rotationally-fixed manner in the housing. The propulsion element may be coupled via a thread to the drive element and may move exclusively axially in the discharge direction and not, for example, in a screwing propulsive movement. As a result, friction between a distal end face of the propulsion member and the piston may be avoided, and it may, moreover, be simpler to provide the drive element and/or the propulsion element with a changed thread or with a special threaded surface coating than the housing. An only partial initial screw connection of the drive element and the propulsion element can also be used to adapt the starting position of the propulsion element to a wide variety of filling volumes. For this purpose, no variants of the propulsion element may be necessary—in particular, if the discharge process is started by enabling the rotation of the axially-fixed drive element.

Advantageously, the drive element may have a threaded rod with an external thread, and the propulsion element may have a sleeve with the axial guide element and an internal thread adapted to the external thread. The internal thread may extend over a length corresponding to the discharge stroke, and the external thread may be shortened to a few turns at the proximal end of the threaded rod or may include a threaded segment of less than one turn. Alternatively, the external thread may extend over the length of the discharge stroke, and the internal thread may be correspondingly shortened. When the threaded rod rotates, the non-rotatingly mounted sleeve may be pushed in the distal direction, which may be easier to accomplish than the linear guidance of a threaded rod.

The torsion spring as an elastic means for generating a torque may be a spiral spring, but can also be configured as a leaf spring, mainspring, conical spring, helical torsion spring, torsion bar, or combinations thereof. It may be maximally or sufficiently pretensioned for a one-time discharge process of the entire or at least a predetermined content of the product container when the autoinjector is delivered, or before the autoinjector is put into operation. Accordingly, the autoinjector may not have a dose selection mechanism. A pre-filled, disposable, ready-to-use syringe may include the product container and an injection needle non-detachably fastened thereto, and may be held in an axially-fixed manner in the housing of the autoinjector. The autoinjector, or at least the ready-to-use syringe and the syringe holder, may accordingly be provided only for one-time use.

In some embodiments, the autoinjector may include an optical, acoustic, or tactile signaling or indicator unit which may be electronically controlled or activated by the processor unit. This may be done after determining an axial piston position or at least a cumulative rotational movement of the drive element which corresponds to an at least approximately complete discharge, and/or after an absence of a rotation sensor signal may be registered as the end of the discharge process, and after subsequent expiration of a predetermined holding time. The signaling unit may generate a signal which indicates an end of the injection and confirms to the user that the autoinjector can now be safely moved away from the puncture site. This signal may be acknowledged and terminated at the latest when the autoinjector may be removed from the puncture site. The holding time may be typically a few seconds, e.g., between 2 and 15 seconds, and/or between 3 and 10 seconds, and may ensure that the injected amount of medication is completely absorbed by the subcutaneous tissue, and no liquid passes through the puncture site onto the tissue surface after the autoinjector has been moved away. Accordingly, the autoinjector may not have a purely mechanically-triggered end click, in which a stop element may be accelerated by a spring; in particular, after the discharge end has been determined, the discharge spring may not be expanded any further; it may, for this purpose, be fixedly supported on the housing in the expansion direction at the latest after the beginning of the discharge process. This may prevent a component that is arranged between the discharge spring and the housing, such as the stop element, from being loaded with a maximum spring force over a long time, and in particular during a storage period.

In some embodiments, the autoinjector includes a needle protective sleeve which, in a securing movement during removal of the autoinjector from the puncture site, may be moved by a needle protection spring from a proximal end position to a distal end position in order to cover an injection needle. A needle protective sleeve detector may be configured to detect this securing movement, or at least the fact that the needle protective sleeve is no longer in the proximal end position. At the time of detection of the securing movement, the processor unit may determine a piston position or an associated amount of liquid—either a residual amount of liquid in the product container or an amount discharged up to this time point. At least in the case of a determined residual amount above a maximum residual amount, the former may be stored by the processor unit and/or indicated to a user. As a result, it may be possible, after the injection has been interrupted by premature removal of the autoinjector from the puncture site, to determine which amounts of medication have actually been discharged into the tissue, and corresponding measures can be taken.

In some embodiments, the rotation sensor may include an actuator that is rotationally asymmetrical and rotates with the drive element and may be in the form of a gear wheel or a diaphragm with teeth and/or recesses distributed over a circumference. The rotation sensor may further include a sensing element for sensing or detecting the rotating teeth and/or recesses. At least two teeth or recesses may be provided and uniformly distributed over the circumference, where several sensing elements arranged at an offset over the circumference may, alternatively or additionally, be provided. In a first variant, the rotation sensor may include a sensing element that is mechanically deflectable, radially or axially, through the teeth and/or recesses. The sensing element may include an electromechanical switch or a strain gauge, where a lever of the switch or a flexible carrier of the strain gauge may be oriented axially or radially. In a second variant, the rotation sensor may include a photosensor, which may act in a contact-free manner, with a light source and a photodetector acting as a sensing element for detecting light of the light source propagating in parallel to the longitudinal axis through the recesses of the diaphragm.

In some embodiments, the rotation sensor may include a rotationally-asymmetrical surface that rotates with the drive element and is subdivided into sectors, where an optical reflection behavior between the sectors changes. The rotation sensor may include a photosensor, which operates in a contact-free manner, with a light source and a photodetector acting as a sensing element for detecting light of the light source, which is reflected by a sector of the surface in the direction of the photodetector. The at least two reflecting sectors may have a bright color or be designed to be reflective. An exemplary subdivision of the surface into 8 sectors means a corresponding resolution of the rotational position of 45°. The surface may belong to a flange of the spring coil or to a disk or another body with an axis of rotation parallel to the longitudinal axis.

In some embodiments, the rotation sensor may include a contactless electromagnetic sensor and a discrete configuration, rotating with the drive element, of passive electromagnetic actuators. These include, for example, a Hall sensor together with a rotatable configuration of permanent magnets, or an inductive sensor together with a rotatable configuration of inductively-detectable electrical conductors.

In some embodiments, the autoinjector may include a communications unit for communicating with a mobile or stationary third-party device and/or an indicator unit for indicating a state of the autoinjector. The processor unit, the communications unit, and/or the indicator unit, and an energy source for supplying them may be arranged on a printed circuit board, which, together with a housing part that can be easily separated (e.g., via predetermined break lines in the housing part), may be removed from the rest of the autoinjector. In some examples, all disposal-critical electronic components of the rotation sensor may also be arranged on the printed circuit board, so that no electronic components remain in the autoinjector after the printed circuit board has been removed.

In some embodiments, the autoinjector includes a device cap, a needle protective sleeve, and a needle protection spring, where, in a delivery or initial state, the needle protective sleeve may be pushed into a first stop by the needle protection spring. After removal of the device cap, but before the discharge process, in an unlocked state ready for injection, the needle protective sleeve may be pushed by the needle protection spring into a second stop with the housing, the syringe holder, or an axially-fixed mechanical holder so that the needle protective sleeve may be located in a front end position further distally than in the delivery state. The autoinjector may further include an axial position detector for detecting this initial movement of the needle protective sleeve in the distal direction into the second stop, or for detecting the needle protective sleeve in the second stop. As a result, a device cap removal may be detected indirectly, e.g., not directly on the device cap itself, and in particular by a detector arranged in the proximal or rear half of the autoinjector. The distal half of the autoinjector, and in particular the region of the ready-to-use syringe, may thus remain free of electronic components and/or electrical conductors. Alternatively, the presence/absence of an excessively long device cap extending into the proximal half of the autoinjector may be detected directly with a cap detector arranged in or on the proximal device half.

The completed device cap removal may be suitably signaled to the user, and/or a sensor unit, such as said rotation sensor or another propulsion sensor in the case of a linear drive by a compression spring may be activated by the detected device cap removal so that it is ready for an immediately imminent use and does not have to be activated permanently or be activated only by the movement to be detected itself. If the axial position detector is an electromechanical switch, a contact may be closed by moving a contact element or tilting of a switching lever, which may be recognized by a processor element in the energy-saving mode and may be used to activate previously currentless components or parts. Electronic components of an electronics module of the autoinjector, which are activated as described in the present disclosure, may thus be operated overall in an energy-saving manner over their life cycle, including the storage period and the use during an injection. A battery or other energy storage unit for their supply may be dimensioned to be correspondingly small. After the device cap removal has been detected, the electronics module may carry out a comprehensive self-test, including, for example, a check of the date, treatment plan, temperature history, current temperature, user information, and/or status of a communications connection.

In some embodiments, upon contact and while pressing the needle protective sleeve onto an injection site in the proximal direction relative to the housing, and while tensioning the needle protection spring, the needle protective sleeve may be moved into a rear end position, thereby starting or enabling a discharge process. Optionally, a signal of the axial position detector generated on this occasion or a first signal of the rotation sensor may be defined and registered as the start of the discharge process. At the end of the injection, when the autoinjector is moved away from the injection site again, the needle protective sleeve may move again in the distal direction into a needle protection position, which can coincide with the first end position. In some examples, the axial position detector may also detect this final movement of the needle protective sleeve into the needle protection position, from which the processor unit can determine whether the holding time was correctly maintained after the end of the discharge process.

An autoinjector according to the disclosure includes a housing, a ready-to-use syringe or other product container arranged in an axially-fixed manner in the housing, a discharge spring, which may be pre-loaded when the autoinjector is delivered, in the form of a compression or torsion spring for the one-time and complete discharge of an amount of liquid from the product container, a propulsion element driven by the discharge spring for the propulsion of a piston in the product container, a sensor unit for determining an axial piston position of the piston in the product container, and a signaling unit, which may be controlled by a processor unit after determining an axial piston position corresponding to an at least approximately complete discharge and after subsequent expiration of a predetermined holding time, for signaling an end of the injection. One end of the discharge spring may be supported on the housing; the discharge spring may expand, for the propulsion of the piston. A proximal end of a helical or compression spring, or an outer end of a spiral spring, may be fixedly connected to the proximal end cap of the housing. This may prevent a component, arranged between the discharge spring and the housing, such as a mechanical stop element, from being loaded with a maximum spring force over a long time, and in particular during a storage period. The end of the discharge process or the beginning of the holding time may be indicated via the electronic signaling unit or may also be completely omitted, in some examples. As a result, a user may not be confused by a further signal prior to the delayed signal, which indicates to the user the end of the injection, including holding time, and allows the removal of the autoinjector from the injection site.

Accordingly, an autoinjector with a housing, a product container, a discharge spring for the one-time and for instance complete discharge of an amount of liquid from the product container, a propulsion element driven by the discharge spring, a sensor unit for determining an axial piston position of a piston in the product container, and a signaling unit, which may be controlled by a processor unit after determining an axial piston position corresponding to an at least approximately complete discharge and after subsequent expiration of a predetermined holding time, for signaling an end of the injection, may be characterized in that one end of the discharge spring is supported on the housing.

### BRIEF DESCRIPTION OF THE FIGURES

FIG. 1 depicts an autoinjector with an electronics module in accordance with embodiments of the present disclosure.

FIG. 2 depicts a first rotation sensor with a fork light barrier in accordance with embodiments of the present disclosure.

FIG. 3 depicts a second rotation sensor with a reflection light barrier in accordance with embodiments of the present disclosure.

FIG. 4 depicts a third rotation sensor with an electromechanical switch in accordance with embodiments of the present disclosure.

FIG. 5 depicts a fourth rotation sensor with a Hall sensor in accordance with embodiments of the present disclosure.

FIGS. 6a, 6b and 6c depict an autoinjector with an electronics module and axial position detector in accordance with embodiments of the present disclosure.

FIG. 7 depicts a first axial position detector with an electromechanical switch in accordance with embodiments of the present disclosure.

FIG. 8 depicts a second axial position detector with a fork light barrier in accordance with embodiments of the present disclosure.

FIGS. 9a and 9b depict a third axial position detector with a reflection light barrier in accordance with embodiments of the present disclosure.

FIG. 10 depicts a fourth axial position detector with a reflection light barrier in accordance with embodiments of the present disclosure.

FIG. 11 depicts a fifth axial position detector with a sliding contact sensor in accordance with embodiments of the present disclosure.

FIGS. 12a and 12b depict two autoinjectors with a break line in accordance with embodiments of the present disclosure.

### DETAILED DESCRIPTION

The term, “product,” “medication,” or “medicinal substance,” in the present context includes any flowable medicinal formulation that is suitable for controlled administration by means of a cannula or hollow needle into subcutaneous or intramuscular tissue, e.g., a liquid, a solution, a gel, or a fine suspension containing one or more medicinal active ingredients. A medication can thus be a composition with a single active ingredient or a premixed or co-formulated composition with several active ingredients from a single container. The term includes in particular drugs, such as peptides (e.g., insulins, insulin-containing medications, GLP-1-containing preparations, as well as derived or analogous preparations), proteins and hormones, biologically obtained or active ingredients, active ingredients based upon hormones or genes, nutrient formulations, enzymes, and other substances, both in solid (suspended) or liquid form. The term can also include polysaccharides, vaccines, DNA or RNA or oligonucleotides, antibodies or parts of antibodies, as well as suitable base substances, excipients, and carrier substances.

The term, “distal,” may refer to a side or direction directed towards the front, piercing-side end of the administration apparatus or towards the tip of the injection needle. In contrast, the term, “proximal,” refers to a side or direction directed towards the rear end of the administration apparatus that is opposite the piercing-side end.

The term, “injection system” or “injector,” may be understood in the present description to mean an apparatus in which the injection needle is removed from the tissue after a controlled amount of the medicinal substance has been dispensed. In contrast to an infusion system, the injection needle in an injection system or in an injector thus does not remain in the tissue over a longer period of several hours.

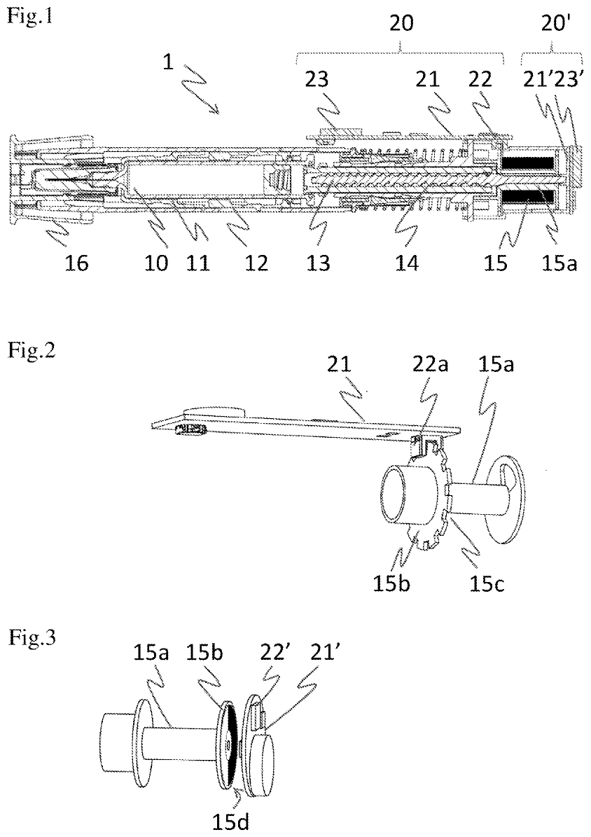

FIG. 1 depicts an autoinjector with an electronics module in accordance with embodiments of the present disclosure. FIG. 1 shows a partial longitudinal section through an autoinjector 1 along its longitudinal axis, including a ready-to-use syringe 10, a syringe holder 11, a needle protective sleeve 12, a drive element 13 in the form of a threaded rod, a propulsion element 14 in the form of a sleeve with an internal thread in engagement with the thread of the threaded rod, and a torsion spring in the form of a mainspring or spiral spring 15 that may be wound on a spring coil 15a. The spring coil 15a may be coupled in a rotationally-fixed and axially-fixed manner to the drive element 13. The spring coil and drive element can thus also be formed in one piece. Instead of a device housing that surrounds the aforementioned components on all sides and accommodates in an axially-fixed manner at least the syringe holder 11 and the mainspring, a device cap 16, which may be pulled off of the autoinjector together with a needle protective sleeve before the administration, is shown. An electronics module 20 with a lateral printed circuit board 21 that is arranged laterally next to the drive element 13, as well as an alternative electronics module 20′ with a proximal printed circuit board 21′ that is arranged proximally of the torsion spring 15, are shown schematically. Alternative rotation sensors 22 may be mounted on the printed circuit boards, as well as a processor unit and a communications unit including an energy source 23, 23′ for supplying them. The printed circuit boards may be fixedly arranged within the device housing of the autoinjector.

FIG. 2 depicts a first rotation sensor with a fork light barrier in accordance with embodiments of the present disclosure. FIG. 2 shows, in a perspective view, the spring coil 15a, a proximal portion of the lateral printed circuit board 21, and a rotation sensor including a fork light barrier 22a arranged on the printed circuit board. The spring coil may include a distal flange 15b in the form of a diaphragm, arranged concentrically and perpendicularly to the longitudinal axis, with a radial modulation 15c of the flange circumference in the form of recesses or indentations arranged and distributed over the circumference. The fork light barrier 22a may be configured such that light from an integrated light source on an inner side of the fork can reach an integrated light detector on an opposite inner side via a light path. The light barrier may be placed with the light path in the longitudinal direction of the autoinjector such that, with a corresponding orientation, the light path traverses one of the recesses or may be interrupted by a peripheral segment, separating two adjacent recesses in the peripheral direction, of the diaphragm. A rotation of the diaphragm, and thus of the spring coil 15a and of the drive element, may be detected by a continuous sequence of light pulses impinging on the light detector, which may be counted and converted into a cumulative rotational movement.

As an alternative to the distal flange, a proximal flange of the spring coil could also be used for rotation detection, or, otherwise, a disk connected to the drive element in a rotationally-fixed manner. Instead of the recesses shown on the periphery of the diaphragm, slits or holes arranged on an axis-concentric circle in the diaphragm may also allow the light to pass along the light path or cover the light detector in a manner dependent upon the angle of rotation. If the light path of the light barrier is oriented radially, the light path may be interrupted by a pinnacle- or crown-shaped closure of a cylinder sleeve with an axis parallel to the longitudinal axis. The number of recesses, slits, or pinnacles may define the resolution of the rotation detection. A configuration with only one recess, one slit, or one pinnacle already generates two transitions or changes in the signal of the detector, and thus distinguishes two rotational positions per revolution of the drive element, resulting in a resolution that is improved in comparison with a detection of complete revolutions. The light source may emit visible light or may include an infrared (IR) LED. The light detector may include a phototransistor or photomicrosensor.

FIG. 3 depicts a second rotation sensor with a reflection light barrier in accordance with embodiments of the present disclosure. FIG. 3 shows, in a perspective view, a proximal end of the spring coil 15a with a proximal flange 15b, the proximal printed circuit board 21′, and a rotation sensor 22′ arranged on the circuit board and includes a reflection light barrier. A pattern 15d with a light-absorbing, dark-colored and a light-reflecting, light-colored sector may be applied to the proximally-directed side of the flange. The rotation sensor may include a light source and a light detector for detecting the light emitted by the light source and reflected by the light-colored sector. The illustrated configuration may not, radially, require any additional space.

Instead of white light, the light source may also emit light of a specific wavelength or wavelength range, including IR, and the reflecting sector may be colored with color of the same wavelength. As an alternative or in addition to a different coloration of the sectors, the proximally-directed side of the flange may also have an alternating surface quality (rough and smooth), or differently-inclined mirror surfaces to guide the light in each case to the detector or in a different direction. The surface portions with different reflection properties may also be attached to an inner or outer surface of a cylinder sleeve with an axis parallel to the longitudinal axis, where the reflection light barrier may be oriented radially from the inside or outside onto the rotating pattern 15d.

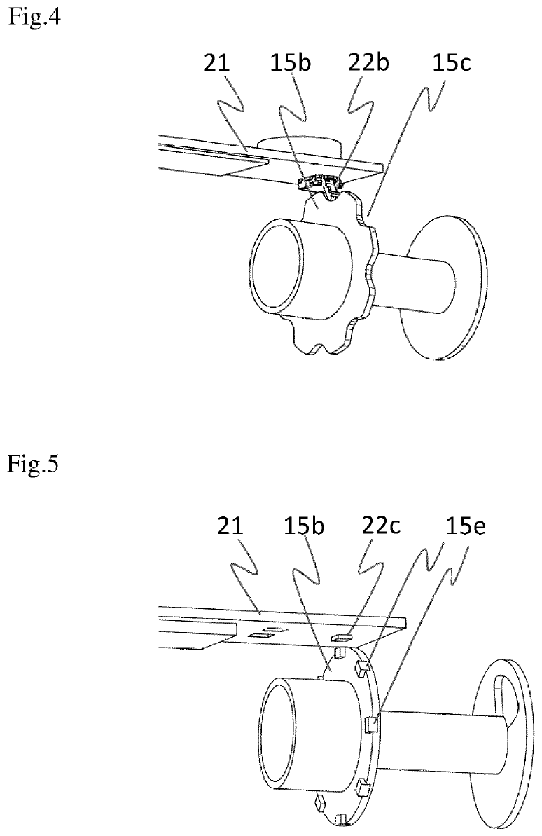

FIG. 4 depicts a third rotation sensor with an electromechanical switch in accordance with embodiments of the present disclosure. FIG. 4 shows, in a perspective view, the spring coil, a proximal portion of the lateral printed circuit board 21, and a rotation sensor including an electromechanical switch 22b. The distal flange 15b of the spring coil may include a radial modulation 15c of the flange circumference in the form of waves or teeth, which may radially tilt a lever of the switch 22b during a rotational movement. After triggering, the lever may autonomously return to its unactuated position and may thus ready for a next triggering. As an alternative to the modulation of the flange circumference, radially-oriented ribs or depressions may be attached to an end face of the distal flange 15b of the spring coil, which in turn tilt an axially-deflectable lever of a switch during a rotational movement.

Instead of the electromechanical switch, it may also be possible to use a correspondingly-positioned strain gauge for detecting a rotational movement of the structures of the respective flange. Each individual rib or each indentation of the flange may press one end of the strain gauge tangentially to the side or radially outwards, so that the resulting mechanical stress in the strain gauge may be detected. The strain gauge itself may be arranged on an arm at the proximal end of the printed circuit board, which is oriented axially or tangentially and may thereby be deflected radially or perpendicularly to the plane of the circuit board. The arm may be formed by recesses or slits in the carrier material of the printed circuit board, where the flexibility of the arm may be controlled. Instead of the strain gauge, a piezo element may also be provided, which may be suitably compressed or stretched by the deflection of the arm.

FIG. 5 depicts a fourth rotation sensor with a Hall sensor in accordance with embodiments of the present disclosure. FIG. 5 shows, in a perspective view, the spring coil, a proximal portion of the lateral printed circuit board 21, and a rotation sensor including a Hall sensor 22c. Eight permanent magnets 15e, the presence of which or the passage of which is detected by the Hall sensor 22c on the circuit board, are arranged and distributed over the circumference on the distal flange 15b of the spring coil. Instead of the discretely-arranged magnets, a permanent magnetic ring with differently-oriented magnetic segments may also be provided. In addition or alternatively, discretely-configured, magnetizable elements or a ring of magnetically-active material with a thickness modulated over the circumference may be provided, in each case together with a permanent magnet that is fixedly placed next to the Hall sensor and the magnetic field, which may be influenced at the location of the sensor by the presence/absence of the magnetizable material. Instead of or in addition to the Hall sensor 22c, a Reed switch or a magneto-resistive resistor may be implemented. A rotation sensor based upon sliding contacts and rotating conductor portions may also be implemented.

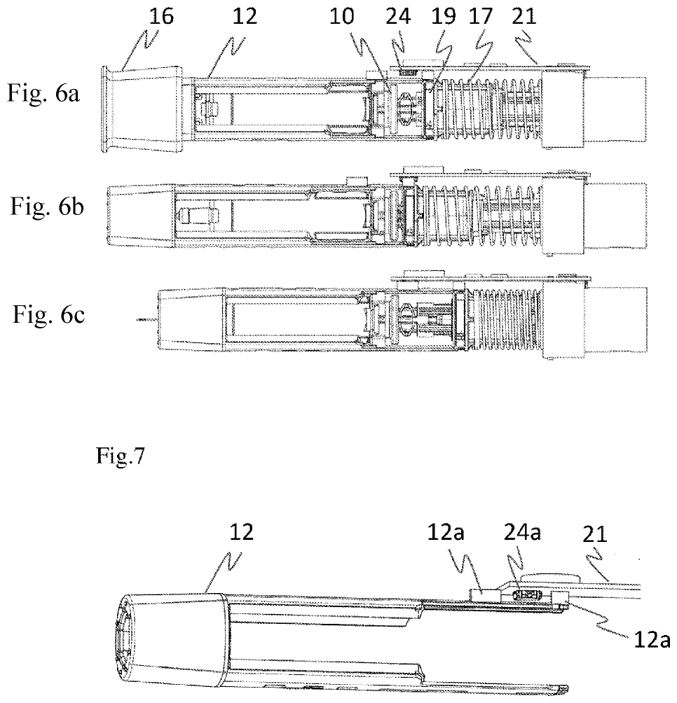

FIGS. 6a, 6b and 6c depict an autoinjector with an electronics module and axial position detector in various stages of use in accordance with embodiments of the present disclosure. These figures show a view of an autoinjector according to a second aspect of the disclosure, where the device housing has again been omitted. The device cap 16, the needle protective sleeve 12, a finger flange of the ready-to-use syringe 10, the needle protection spring 17, and a lateral printed circuit board 21 with an axial position detector 24 mounted thereon for the needle protective sleeve can be seen. At FIG. 6a, the autoinjector may be in a delivery state, and the needle protective sleeve 12 may be in a starting position. The device cap 16 may be anchored in an axially-fixed manner on the housing of the autoinjector by means of suitable cams or releasable catches. The needle protection spring 17 presses a first, distally-directed, rigid surface or edge of the needle protective sleeve into a first stop, which may be formed directly by the device cap 16. The first stop may also be provided on the housing, on the syringe holder, or on a mechanical holder of the autoinjector and may have a stop surface that is oriented to be oblique, not perpendicular, to the longitudinal axis. In this case, a radially-flexible locking element of the needle protective sleeve may be in engagement with the first stop and may be secured in this engagement by the mounted device cap. This may prevent the force of the needle protection spring 17 from acting on the device cap 16 in the delivery state and leading to an undesired movement of the needle protection cap 16, and thus to an impairment of the sterility of the needle. After removal of the device cap 16, the locking element may be pushed, via the oblique stop surface, out of engagement. If the needle protective sleeve 12 is non-positively snap-fitted to a switching sleeve 19 or is even formed in one piece therewith, the switching sleeve 19, instead of the needle protective sleeve 12, may also be pushed into the first stop via the distal end of the needle protection spring 17, as described.

With reference to FIGS. 6b and 6c, after a removal of the device cap 16 with release of the cams or catches, the needle protective sleeve 12 may move distally through the expanding needle protection spring 17. For instance, at FIG. 6b, the autoinjector may be in an unlocked state ready for injecting and discharging. The needle protection spring 17 may press a second, distally-directed surface of the needle protective sleeve 12 or of the switching sleeve 19 against a second stop of the housing; the needle protective sleeve 12, may be located in a distal end position. The second stop may also be formed by the syringe holder, the mechanical holder, or, otherwise, by a component attached in an axially-fixed manner to the housing.

For injection, the distal end of the needle protective sleeve 12 may be pressed against the puncture site, as a result of which the needle protective sleeve 12 may be displaced in the proximal direction into and relative to the housing while the needle protection spring 17 is being compressed. In order to adjust a piercing depth (e.g., in a range of 5 to 8 mm), and in particular to shorten it, suitably attached, short axial ribs or projections on one of the two stop components may define the proximal stop of the needle protective sleeve 12 on the housing, or the proximal stop of the switching sleeve on the mechanical holder. At the same time, the trigger elements that are attached to the switching sleeve or interact with the switching sleeve may to be positioned in an axially-adjustable manner for triggering the discharge. With reference to FIG. 6c, the autoinjector may be in an injected state, with the needle protective sleeve 12 in a rear end position. After the discharge process has been completed, the autoinjector may be lifted off the puncture site, whereby the needle protective sleeve 12 again moves distally up to the front end position, and may then be secured against re-insertion by a securing mechanism.

The axial position detector 24 may detect the needle protective sleeve 12 in the front end position and in the rear end position. Alternatively to the detection of the axial end positions, an axial movement or the transition of the needle protective sleeve 12 from the starting position to the front end position or from the front end position to the rear end position may also be registered. If the syringe 10 should move distally relative to the housing by a piercing mechanism itself, the needle protective sleeve 12 in a final end position in the secured state may be further forward than before. Instead of the needle protective sleeve 12, the switching sleeve 19 or a further sleeve, which may be connected in an axially-fixed manner to the needle protective sleeve, may also be detected in its end positions or the movements in-between. In this case, the functionality of the axial position detector may be tested by moving the switching sleeve 19 in a partially manufactured state of the autoinjector, before a drive unit with the switching sleeve 19 and the electronics module and a syringe unit with the needle protective sleeve 12 and the syringe 10 are finally assembled. If the needle protection spring, unlike in FIGS. 6a-6c, is arranged radially within the switching sleeve 19, the axial position sensor 24 may thereby be placed even further proximally, and in particular axially, at the height of the foremost position, shown in FIG. 6b, of the proximal end of the switching sleeve.

The first detection of the front end position may cause electronic components of the electronics module 20—in particular, the rotation sensor 22 discussed above—to be switched on, activated, and/or woken up. If the axial position detector is an electromechanical switch, an electrical contact may be closed for the first time by moving a contact element—in particular, by tilting a switching lever caused by mechanical contact with a first protrusion or formation on the needle protective sleeve 12. This may be recognized by a processor element in a power-saving, deep-sleep mode, in which the processor element monitors the switch even over a storage period (e.g., of up to several years). By means of the processor element, further components may be subsequently connected to the energy storage unit or otherwise activated. Alternatively, by tilting, a switching lever may be used directly to close an electrical contact in a supply circuit, where previously currentless components of the electronics module may be switched on. Further alternatively, a supply circuit may also be closed by moving an insulator that electrically separate two contact points mechanically pretensioned against one another.

FIG. 7 depicts a first axial position detector with an electromechanical switch in accordance with embodiments of the present disclosure. FIG. 7 shows, in a perspective view, a distal portion of the lateral printed circuit board 21 and an axial position detector including an electromechanical switch 24a. The switch 24a may be positioned axially such that a lever of the switch is tilted in opposite directions by two protrusions 12a or ribs on one arm of the needle protective sleeve 12 in the end positions of the needle protective sleeve. The placement of the two protrusions 12a may be at a distance corresponding to a stroke of the needle protective sleeve 12 between the end positions, which may enable the use of a single, bidirectional switch for detecting two states. The lever of the illustrated switch 24a may return autonomously to its unactuated position after a first triggering or tilting, and may thus be ready for a next triggering in one of the two directions. The protrusions 12a may also be attached to a switching sleeve —such as when they are snap-fitted in an axially-fixed manner to the needle protection spring.

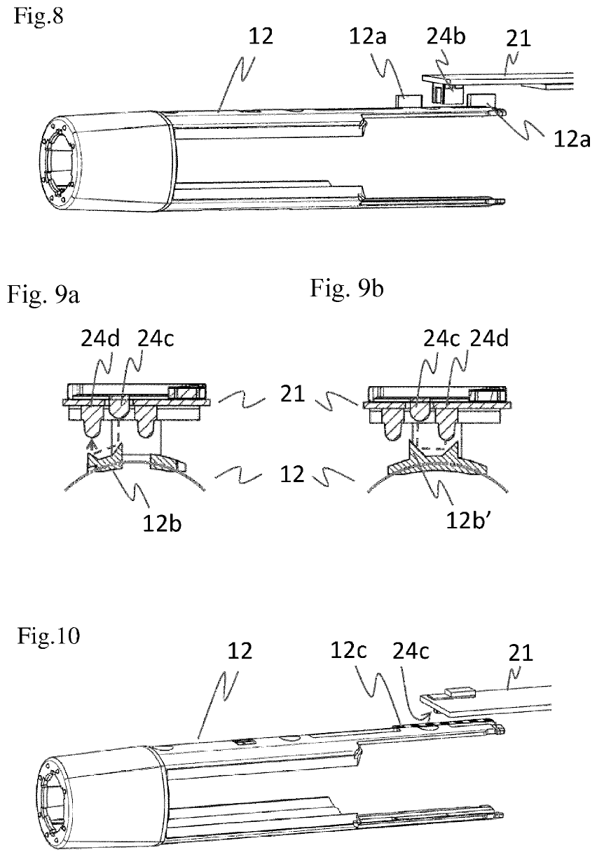

FIG. 8 depicts a second axial position detector with a fork light barrier in accordance with embodiments of the present disclosure. FIG. 8 shows, in a perspective view, a distal portion of the lateral printed circuit board 21 and an axial position detector including a fork light barrier 24b. The light barrier may be interrupted by two protrusions 12a or ribs on one arm of the needle protective sleeve 12 in the end positions of the needle protective sleeve. For this purpose, the protrusions 12a may be at an axial distance corresponding to the stroke of the needle protective sleeve 12. A differentiation between the two end positions may be possible with a second light barrier.

FIGS. 9a and 9b depict a third axial position detector with a reflection light barrier in accordance with embodiments of the present disclosure. FIGS. 9a and 9b each show a section through the autoinjector at the height of an axial position detector including a reflection light barrier, with the needle protective sleeve 12 in a first end position (FIG. 9a) and in a second end position (FIG. 9b) of the needle protective sleeve. A light beam from a light source 24c may be deflected towards two different light detectors 24d by two, differently-oriented mirrors 12b, 12b′ on the arm of the needle protective sleeve 12 in the end positions. The light source may emit visible light or include an IR LED. One or more of the light detectors may include a phototransistor tuned to the wavelength of the light source.

FIG. 10 depicts a fourth axial position detector with a reflection light barrier in accordance with embodiments of the present disclosure. FIG. 10 shows, in a perspective view, a distal portion of the lateral printed circuit board 21 and an axial position detector including a reflection light barrier. A pattern 12c with light-absorbing, dark-colored and light-reflecting, light-colored portions may be applied to an outward-directed side of an arm of the needle protective sleeve 12. The reflection light barrier may include a light source 24c and a light detector for detecting the light emitted by the light source and reflected by a light-colored portion. As an alternative or in addition to a different coloration of the portions, the latter may also have an alternating surface quality, or differently-inclined mirror surfaces, which may direct the light in each case to the detector or in a different direction. Corresponding to an axial extension of the portions, the distance of a displacement of the needle protective sleeve may be measured with a desired resolution, from which the assumption of an end position can be identified.

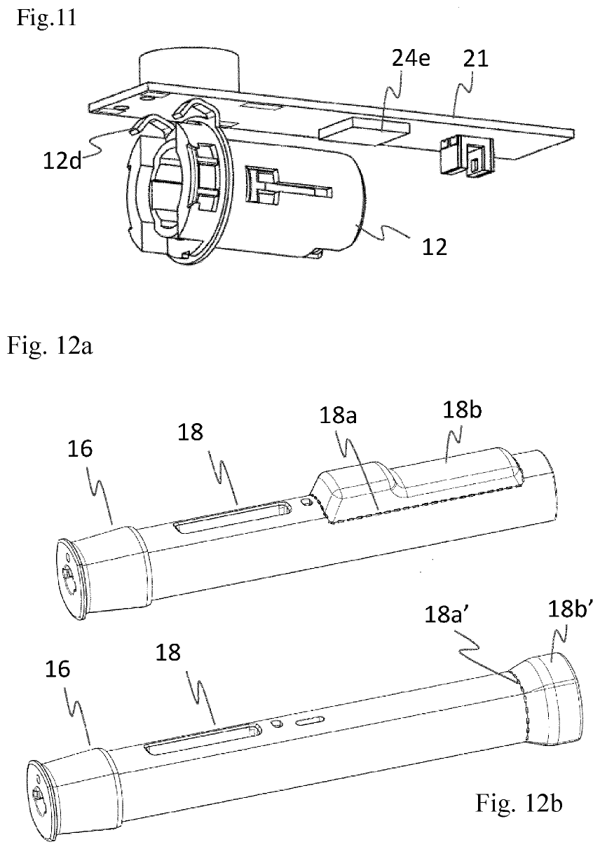

FIG. 11 depicts a fifth axial position detector with a sliding contact sensor in accordance with embodiments of the present disclosure. FIG. 11 shows, in a perspective view, a portion of the needle protective sleeve 12, a lateral printed circuit board 21, and an axial position detector including a sliding contact sensor 24e. In the present case, the sliding contact may include a sliding element 12d that is connected or coupled in an axially-fixed manner to the needle protective sleeve 12 and has two flexible arms, which, in specific axial positions, may make contact with contact points on the underside of the printed circuit board and establish a conductive connection between them. Alternatively, three sliding elements may also be implemented, of which one may permanently contact an axial conductor track on the underside of the printed circuit board, and the other two, respectively, may contact portions or points of a second and a third conductor track. The portions may be arranged in such a way that a detectable electrical contact exists alternatingly between the first and the second conductor tracks and between the first and the third conductor tracks. In this example, the distance of a displacement of the needle protective sleeve may be measured with a resolution corresponding to the axial extension of the portions, from which the assumption of an end position may be identified. Further alternatively, a resistance element with an axial extension corresponding to at least the stroke of the needle protective sleeve may be contacted, either continuously or in discrete steps, by the sliding contact. The resistance element may also be printed onto the printed circuit board and may have a high electrical resistance in comparison to the other conductor tracks, so that a measuring circuit with a measurable, axial position-dependent resistance may be formed via the sliding contact. As a result, an absolute position of the sliding contact and the needle protective sleeve may be determined.

FIGS. 12a and 12b depict two autoinjectors with a break line in accordance with embodiments of the present disclosure. FIGS. 12a and 12b show a perspective views of two autoinjectors according to a further aspect of the disclosure. The housing 18 of the autoinjectors may have a separating or break line 18a, 18a′ along which a peripheral housing part 18b, 18b′ can be irreversibly separated from the rest of the housing. An electronics module, optionally including one of the printed circuit boards shown in FIG. 1 and arranged laterally or proximally with at least the processor unit and an energy storage unit for supplying them, may fixedly connected to the separable housing part 18b and coupled via further break points or form-fitting contact points to other components of the autoinjector. A separation of the peripheral housing part may thus remove the electronics module from the rest of the autoinjector, such that it may be supplied to a disposal separate from the mechanical and/or medical components of the autoinjector. In some examples, the electronics module may include all the disposal-critical electronic components of the autoinjector according to the disclosure, so that no electronic components remain in the autoinjector after the electronics module has been separated. Electronic conductor paths or wires, which may be contacted in a form-fitting manner via sliding or plug contacts, in the autoinjector outside the printed circuit board, such as the sliding element of FIG. 11, or actuators based upon shape-memory wires may not be regarded as electronic components in some examples. For this purpose, the electronics system primarily includes sensors that can be removed from a counterpart or non-electronic trigger element even without the exertion of force. The break lines in the housing of the autoinjector may be brought about, for example, by perforation and/or targeted weakening of the housing thickness, and holding or gripping positions may also be provided, e.g., ribs and/or knobs, so that a user can separate a smaller part of the housing together with the printed circuit board anchored therein from a remaining part of the housing without using tools and with only the thumb and fingers of one hand, and can dispose of it as intended. For example, a printed circuit board provided at the proximal end may be twisted off by a rotating or screwing movement of a proximal end cap in relation to the housing, as is known from the opening of sealed beverage bottles.

As an alternative to the break lines, the peripheral housing part to be separated may also be a component that is different from the rest of the housing, but detachably snap-fitted thereto, so that, after releasing the snap-fit connection, the peripheral housing part may be removed directly and without further application of force. For this purpose, the housing may have an extension or expansion beyond the contour of the separating line in the peripheral direction corresponding to extension or expansion in the snap-fitted state, surrounded as a whole by the peripheral housing part. This housing expansion may carry individual snap-in elements that engage from the interior into recesses in the peripheral housing part. It may also be possible for the housing parts to not be separated immediately after injection by the patient, but instead at a collection point, where suitable tools may also be used. Further alternatively, the peripheral housing part may remain connected to the rest of the housing part by a hinge after the opening of the separating line or the snap-fit connection, and the electronics module, subsequently, may be directly gripped and removed separately. A lock to be released by the processor unit at the end of the injection or, mechanically, during removal of the autoinjector from the injection site, may prevent the proposed separation from occurring too early.

The aforementioned separating or break lines may be different than the boundaries or contours between two housing parts of an autoinjector, which can be assembled from two subunits or assemblies for easier assembly. In this case, a distal syringe unit of the autoinjector may include a first, distal housing part, the needle protective sleeve, the device cap, and the syringe holder, while a proximal drive unit includes a second, proximal housing part, the mechanical holder, the needle protection spring, locking and switching sleeve, drive and propulsion element, and the one-time chargeable energy store for the automatic substance delivery. The proximal housing part may also be referred to as an end cap. In a filling or assembly process, the ready-to-use syringe may be inserted into the syringe unit, and the two subunits may be subsequently assembled, where the two housing parts snap-fit non-detachably and form a boundary line on their surface. The previously-defined electronics module may be part of the drive unit. The proximal housing part may accordingly include the separating or break line and the separable housing part 18b completely.

The ready-to-use syringe 10 may include a cylindrical syringe body as a product container, in which a product-receiving space is delimited between a syringe shoulder and a piston or plunger displaceable along the longitudinal axis. A hollow injection needle may be fixedly connected to the syringe shoulder at a distal end of the syringe body, and a finger flange may be attached to a proximal end of the ready-to-use syringe and projects radially outwards beyond the outer circumference of the syringe body. In the delivery state, the product-receiving space contains a product to be dispensed, which may be displaced entirely or partially out of the product-receiving space through the injection needle by a displacement of the plunger from a plunger start position to a plunger end position.

The injection needle of the ready-to-use syringe 10 may be covered by a needle protection cap, which may be configured as a so-called rigid needle shield (RNS) and includes a rubber-elastic needle protection element and a sheath made of hard plastic. The needle protection cap may protect the injection needle against mechanical effects and contamination, and may keep the injection needle and the product sterile. At the distal end of the autoinjector, in the delivery state thereof, a device or pull-off cap 16 may be arranged to be axially pulled off and/or twisted off and completely removed before the autoinjector is used. The device cap 16 may also have snap hooks, which the needle protection cap may be released from the ready-to-use syringe. The syringe holder may include two elastic fingers, which may be fastened at their proximal ends to a holder sleeve of the syringe holder, and each may have, at their distal ends, an axial support element for the syringe shoulder.

The injection needle may be surrounded by a needle protective sleeve 12 that is mounted in an axially-displaceable manner relative to the housing and may be inserted into the housing. In the starting position of the needle protective sleeve, the distal end of the needle protective sleeve may project distally beyond the needle tip of the injection needle so that access to the needle tip is initially prevented. At its distal end face, the needle protective sleeve may have an opening through which the injection needle can pass and enter an injection site during a relative movement of the needle protective sleeve and the injection needle. The needle protective sleeve may also serve as a trigger element for triggering the product discharge process, where the needle protective sleeve may be displaced relative to the housing in the proximal direction for this purpose, while tensioning a needle protection spring 17. For this purpose, the needle protective sleeve includes two sleeve arms, which, with respect to two recesses of the housing, designated as viewing windows, may be arranged offset or rotated by 90° about the longitudinal axis. After the injection has taken place, the needle protective sleeve may be displaced, relative to the housing, from the actuated position along the longitudinal axis in the distal direction to a needle protection position and may be blocked there from being pushed back again.

The autoinjector may include a switching module with a switching sleeve and a locking sleeve surrounded by the switching sleeve. The switching sleeve may be snap-fitted to a proximal end of the sleeve arms of the needle protective sleeve 12 and may be pushed distally by a distal end of the needle protection spring 17. The needle protection spring may include a spring that is made of metal, acts as a compression spring, and is designed as a helical spring. The locking sleeve may be designed to lock the switching sleeve and the needle protective sleeve, after injection has taken place, in a front end position from being pushed into the housing again. Radially outward-directed projections on the resilient arms of a locking member of the locking sleeve may engage behind a proximal edge of the switching sleeve so that the switching sleeve and thus the needle protective sleeve, relative to the locking sleeve, cannot move in the proximal direction. The locking sleeve may be at a minimum distance from a distally-directed, axially-fixed end face of the autoinjector, so that the locking sleeve may move in the proximal direction at most by significantly less than the distance between the injection needle tip and the distal end of the needle protection spring. The locking may be achieved by a proximal locking stroke of the locking sleeve relative to the switching sleeve, in which stroke the locking member may be detached from the switching sleeve for movement inwards and engages behind a proximally-directed edge of the autoinjector by the spring action of the arms. When the autoinjector is subsequently removed from the puncture site, the switching sleeve may be pushed by the needle protection spring in the distal direction over the locking member, whereupon the locking member engages behind a proximally-directed edge of the switching sleeve in a locking position by the spring action of the arms and blocks the switching sleeve and the needle protective sleeve from renewed movement in the proximal direction. A correct locking of the needle protective sleeve against renewed insertion may not be detected separately, but may be ensured by the described mechanical sequence control.

The electronics module of the autoinjector may include a printed circuit board and a sensor unit, arranged thereon, for detecting states or processes, a processor unit for processing signals of the sensor unit, a communications unit for wireless communication of data from the processor unit (e.g., to a third-party device), and an energy storage unit for supplying the aforementioned units. The energy storage unit may be sufficiently charged for a storage period of up to several years with minimal power consumption, and for a one-time use of the autoinjector with brief signal processing and data exchange. A battery suitable as an energy storage unit may not be rechargeable, but accessible in a separate battery compartment for separate disposal. The direct communication with a stationary, third-party device, e.g., an expert system in a delocalized or cloud-based infrastructure, for transmitting data of an injection process may take place, for example, via a 5G or 4G/LTE mobile radio network—in particular, a Narrowband Internet-of-Things, NB-IoT, or another suitable means, such as LoRa, Sigfox, or satellite-based communication. Different protocols may be implemented on a dual-mode chip in order to optimize the geographical coverage, with moderate increase in costs and space requirement. The communication with a mobile device, e.g., a mobile phone or smartphone, or with a stationary gateway to a wired network, may take place via a Bluetooth or BLE connection, which may be initiated via an out-of-band pairing. The BLE communication via the mobile device of the user may take place, simultaneously or redundantly, with the direct communication in the mobile radio network, in order to ensure data exchange. The different communications methods may also be provided in a modular manner; for example, a mobile radio communications unit can subsequently replace or supplement a BLE communications unit—together with an adapted energy storage unit, in some examples. In order to accommodate and electrically contact an optional 5G module with an associated energy store and a suitable SIM card, a mechanical mounting in the device housing and a plug with connection to the printed circuit board may be provided by default, so that no components of the autoinjector have to be adapted as a function of a decision for or against a 5G module. Without a 5G module, the mounting and plug may remain unused. The sensor unit may include a temperature sensor for detecting a temperature profile in the autoinjector during a warm-up phase, after the autoinjector is removed from a refrigerator. Heating of the refrigerated medications before subcutaneous injection may reduce the perceived pain; accordingly, it may be helpful if the reaching of a target temperature is signaled to the user. In this case, the temperature sensor does not necessarily have to be mounted in or on the ready-to-use syringe, since a temperature profile at another point, e.g., on the printed circuit board of the electronics module, may be sufficiently meaningful for these purposes. In particular, a time delay of the medication temperature relative to the sensor temperature may demonstrate a reproducible behavior independent of the filling volume of the syringe and the ambient temperature, and can be determined empirically for a specific sensor position. In the case of an almost constant temporal temperature lead of the sensor, a target temperature time point of the medication may be estimated by waiting for a predetermined period of time in the range of 30-90 seconds when the target temperature at the sensor is reached, where the lower value applies to sensor positions in direct proximity to the ready-to-use syringe. Alternatively, in the case of an approximately exponential approach of the sensor temperature to an unknown ambient temperature, a critical temperature gradient may be determined, with which, if fallen below, a sufficient heating of the medication can be deduced. The temperature sensor may be actively switched on by the user when the autoinjector is removed from the refrigerator. In some examples, this may be done by a switch for closing an electrical contact in a supply circuit or by moving an insulator away, which electrically separates two contact points pretensioned mechanically against one another, and/or by removing a tear-off film.

The temperature of the autoinjector or of the medication may be monitored and registered by the temperature sensor over the entire transport chain. Thus, it may be ensured, for example, that a maximum temperature during transport, including the distance to the refrigerator of the user, has not been exceeded, or exceeded at most over an acceptable period of time. For the resource-saving and precise determination of a critical time point when temperature was exceeded in the supply chain, the sensor unit may have a suitable, time-stable, oscillating crystal as a frequency generator or clock generator. The oscillations of the crystal may be counted for the time period from the critical time point up to the moment in which the processor unit is woken up or synchronized, and the critical calendar time point is retroactively determined therefrom. The dedicated oscillating crystal may make it possible to dispense with actively operating a processor with a calibrated clock during the entire transport and storage period. The electronics module may further include an optical, acoustic, and/or tactile indicator unit, such as an optical display, in which an optical waveguide may guide the light of a light source on the printed circuit board to the surface of the housing. A state indicated by the indicator unit may include a device state of the autoinjector, a module state of the electronics module, or a process state of an ongoing or completed injection process. In some examples, the indicator unit of the electronics module may be kept simple and limited to a few LED's, e.g., in traffic light colors or for illuminating selected pictograms, and/or to an acoustic signal generator for generating language-independent sounds or melodies. This may be advantageous particularly in cooperation with the advanced graphical display options and voice output options of a smartphone, since the smartphone coupled wirelessly to the electronics module takes over the refined communication with the user that goes beyond a status display. Alternatively, the indicator unit may be part of a complete human-machine interface, which further has a screen and/or voice output options for informing a user, as well as input options, such as switches, capacitive touch sensors, and/or voice recognition for controlling the units.

The mobile device may be initially set up and configured—for example, by installing an application and registering the user. This can be done by means of a patient data card which, via near-field radio communication (NFC) or optical QR codes, transfers all relevant data to the mobile device. If the mobile device is in the range of the autoinjector, injection data may be transmitted during an injection process in real time. In this case, the mobile device may output instructions to the user in real time, and thus guide the user through the next steps. In any case, the injection information may also be stored in the electronics module and be transmitted only later, in consolidated form. The data received by the mobile device can be supplemented by the user, e.g., by specifying the injection site, and are forwarded to an expert system in a suitable way. The expert system may store the data and supplies patients, medical personnel, and health insurance companies with targeted information, and thus may support compliance with a treatment plan by the user of the injection device.

LIST OF REFERENCE SIGNS

| | |
|---|---|
| 1 | Autoinjector |
| 10 | Ready-to-use syringe |
| 11 | Syringe holder |
| 12 | Needle protective sleeve |
| 12a | Protrusion |
| 12b | Mirror |
| 12c | Pattern |
| 12d | Sliding element |
| 13 | Drive element |
| 14 | Propulsion element |
| 15 | Torsion spring |
| 15a | Spring coil |
| 15b | Flange |
| 15c | Modulation |
| 15d | Pattern |
| 15e | Permanent magnet |
| 16 | Device cap |
| 17 | Needle protection spring |
| 18 | Housing |
| 18a, 18a′ | Separating line |
| 18b, 18b′ | Housing part |
| 19 | Switching sleeve |
| 20, 20′ | Electronics module |
| 21, 21′ | Printed circuit board |
| 22, 22′ | Rotation sensor |
| 22a | Fork light barrier |
| 22b | Switch |
| 22c | Hall sensor |
| 23, 23′ | Energy source |
| 24 | Axial position detector |
| 24a | Switch |
| 24b | Fork light barrier |
| 24c | Light source |
| 24d | Light detector |
| 24e | Sliding contact sensor |

1. An autoinjector comprising:
a housing;
a product container;
a spring for a one-time discharge of a maximum content of the product container;
a drive element;
a propulsion element, wherein, for discharging, the spring is configured to rotate the drive element, and, while rotating, the drive element is configured to produce a propulsive movement of the propulsion element and of a piston in the product container;
a rotation sensor configured for an alternating continuous detection of at least two rotational positions per revolution of the drive element during the discharge process; and
a processor unit configured for determining an axial piston position of the piston in the product container from the detected rotational positions.

2. The autoinjector according to claim 1, wherein the propulsion element has an axial guide element for an exclusively linear propulsive movement in the housing.

3. The autoinjector according to claim 1, further comprising a signaling unit configured to be controlled by the processor unit after determining the axial piston position corresponding to a complete discharge and after subsequent expiration of a predetermined holding time for signaling an end of the discharge process.

4. The autoinjector according to claim 1, further comprising an axial position detector configured for detecting a securing movement of a needle protective sleeve from a rear end position to a front end position for covering an injection needle, wherein the processor unit is configured to store a piston position or a corresponding amount of liquid determined at the time of the detection of the securing movement.

5. The autoinjector according to claim 1, wherein the rotation sensor comprises an asymmetrically configured, rotatable actuator and a sensing element that is configured to be mechanically deflectable or optically coverable by the actuator.

6. The autoinjector according to claim 1, wherein the rotation sensor comprises a rotatable surface with differently reflecting sectors, and a photodetector configured for detecting sectorally reflected light.

7. The autoinjector according to claim 1, wherein the rotation sensor comprises an electromagnetic sensor and a rotatable arrangement of passive actuators.

8. The autoinjector according to claim 1, further comprising a communications unit configured for communicating with a third-party device and/or an indicator unit for indicating a state of the autoinjector.

9. The autoinjector according to claim 8, wherein the processor unit, the communications unit, and/or the indicator unit, and an energy source configured for supplying the units are arranged on a printed circuit board that is configured to be removed from the autoinjector together with a separable housing part.

10. The autoinjector according to claim 9, further comprising a lock that is configured to be mechanically released to allow the separable housing part to be separated from the autoinjector.

11. The autoinjector according to claim 9, wherein the separable housing part is defined by predetermined break lines in the housing.

12. The autoinjector according to claim 9, wherein the separable housing part is configured to be snap-fitted to the housing by a snap-fit connection.

13. The autoinjector according to claim 1, further comprising:
a device cap;
a needle protective sleeve;
a needle protection spring, wherein, in a delivery state, the needle protective sleeve is configured to be pushed into a first stop by the needle protection spring, and, after removal of the needle protection cap, but before the discharge process, the needle protective sleeve is configured to be pushed by the needle protection spring into a second stop; and
an axial position detector configured for detecting a movement of the needle protective sleeve in the distal direction into the second stop.

14. The autoinjector according to claim 13, wherein the axial position detector is arranged in a proximal half of the autoinjector.

15. The autoinjector according to claim 13, wherein the axial position detector is configured to detect a movement of the needle protective sleeve into the second stop from a rear end position, into which the needle protective sleeve is moved in the proximal direction upon contact and while pressing onto an injection site.

16. The autoinjector according to claim 1, further comprising a disposable, ready-to-use syringe comprising the product container and wherein the syringe is held in an axially-fixed manner in the housing of the autoinjector.

17. The autoinjector according to claim 1, further comprising a needle protective sleeve and a needle protection spring, wherein the protective sleeve is configured to be in a distal end position in order to cover an injection needle of the autoinjector, wherein, upon contact and while pressing the needle protective sleeve onto an injection site, the protective sleeve is configured to move relative to the housing into a proximal end position thereby starting a discharge process and wherein, during removal of the autoinjector from the injection site, the protective sleeve is configured to move via the needle protection spring from the proximal end position to the distal end position.

18. A method for preparing an autoinjector for a discharge process, the autoinjector having a device cap, a needle protective sleeve, a needle protection spring, and an axial position sensor, wherein, in a delivery state, the needle protective sleeve is configured to be pushed into a first stop by the needle protection spring, and, after removal of the device cap, but before the discharge process, the needle protective sleeve is configured to be pushed by the needle protection spring into a second stop, wherein the axial position detector includes an electromechanical switch for detecting a movement of the needle protective sleeve in the distal direction to or into the second stop; the method comprising:
detecting movement of the needle protective sleeve in the distal direction into the second stop; and
after detecting movement of the needle protective sleeve in the distal direction, activating a rotation sensor of a sensor unit to detect states or operations of the autoinjector.

19. A drive unit for an autoinjector, comprising:
a spring for the one-time discharge of a maximum content of a product container of the autoinjector;
a drive element;
a propulsion element, wherein, for discharging liquid from the product container, the spring is configured to rotate the drive element, and while rotating, the drive element is configured to produce a propulsive movement of the propulsion element and of a piston in the product container,
a rotation sensor configured for the alternating continuous detection of at least two rotational positions per revolution of the drive element during the discharge process; and
a processor unit configured for determining an axial piston position of the piston in the product container from the detected rotational positions.

20. An autoinjector comprising:
a housing;
a product container;
a torsion spring for a one-time discharge of a maximum content of the product container;
a drive element;
a propulsion element, wherein, for discharging, the torsion spring is configured to rotate the drive element, and the rotating drive element is configured to produce a propulsive movement of the propulsion element and of a piston in the product container;
a rotation sensor;
a processor unit for processing a rotation sensor signal
a separable housing part,
wherein the processor unit and an energy source configured for supplying the processing unit are arranged on a printed circuit board that is configured to be irreversibly removed from the autoinjector together with the separable housing part.
